(19) United States
(12) Patent Application Publication
(10) Pub. No.: US 2011/0288462 A1
(43) Pub. Date: Nov. 24, 2011

(54) HAEMOSTATIC WOUND CARE ARTICLE

(76) Inventor: Birgit Riesinger, Ostbevern (DE)

(21) Appl. No.: 12/672,020

(22) PCT Filed: Aug. 1, 2008
(86) PCT No.: PCT/EP2008/060164

§ 371 Date: Aug. 15, 2011

(30) Foreign Application Priority Data
Aug. 3, 2007 (DE) 10 2007 036 755.6

(51) Int. Cl. A61F 13/00; A61B 17/00; A61P 17/02; A61P 7/04; A61K 47/30; A61K 9/70

(52) U.S. Cl. 602/48; 514/772; 424/445; 606/203

(57) ABSTRACT

The subject matter of the invention is a wound care article exhibiting a quantity of super-absorbent polymers. The wound care article is characterized in that characterized in that it exhibits at least one agent which can restrict the bleeding or the tendency to bleed. In the process it can be a chemically and/or pharmacologically and/or physiologically acting active ingredient or active ingredient complex.

The invention relates to a wound care article exhibiting a quantity of super-absorbent polymers in accordance with the characterizing clause of claim 1.

### STATE OF THE ART

Planar wound dressings have been known for a long time. Planar wound dressings which contain super-absorbent polymers are also well-known. In the case of such wound dressings it can for example be matter of a wound dressing for the absorption of wound exudates, as they are described e.g. in DE10059439 as well as WO03094813 from the applicant of the present invention, the content of said DE10059439 as well as WO03094813 is to be added completely to the disclosure content of the present description.

The named wound dressings however essentially serve the purpose of debridement, the control of excess exudation as well as the mechanical protection of the wound region. The additional admixture of substances which act in a coagulating manner, hemostatic manner or in similar fashion has been unknown up to now.

However, this can be of great significance, since in particular acute wounds oftentimes exhibit profuse bleeding and whose healing is promoted by the above-named properties of the known substances. This also applies for chronic wounds which due to lesions in the case of bandage changes e.g. can tend to bleed freely.

Such cases can occur in the case of accident patients, amputations, iatrogenic wounds or dehiscences. Because the bleeding produces not only a hazard for the affected patient, but rather also constitutes a virtually uncontrollable source of infection for assistants and other people concerned, in particular at the site of the accident. Additionally to be taken into consideration is the fact that uncontrolled bleeding can be the cause for a shock or—in less serious cases—for a considerable psychic crisis of the patient, which can considerably impair the overall constitution or the patient's chances for survival.

In the acute care of bleeding wounds predominantly physical hemostasis continues to be relied upon. In particular the Spanish tourniquet and the tourniquet are used here.

WO99/59647 describes a multi-layer hemostatic bandage which preferably contains one thrombin layer between two fibrinogen layers. The bandage can contain other materials such as glycolic acid or acid-based milk polymers or copolymers.

A hemostatic bandage is also described in WO/97/28832, and likewise exhibits thrombin as well as fibrinogen in a fibroid matrix.

Thrombin and fibrinogen are substances naturally produced by the body, which are for example isolated from blood donations. Their use can pose serological and immunological problems as well as cause infections.

From U.S. Pat. No. 5,800,372 a wound dressing exhibiting a mixture of microfibrillar collagen and super-absorbent polymers is known. Said wound dressing is supposed to absorb blood leaking from a wound and cause coagulation. The employed materials however are rather expensive.

Additional hemostatic bandages are disclosed in U.S. Pat. No. 4,616,644 and EP0206697 A2. Here a thin layer of high molecular polyethylene oxide is used on the surface of a perforated plastic foil. Such materials however do not interact with the naturally occurring hemostatic agents.

From U.S. Pat. No. 6,967,261 a bandage is known which exhibits a cross-linked polyethylene oxide resin (super-absorbent) exhibiting one layer as well as hemostatic agents. In the case of said agents it can be e.g. chitosan-niacinamide-ascorbate, chitosan or sodium alginate.

It is the object of the present invention to provide a wound care article which avoids the disadvantages of the articles described from the state of the art.

These problems are solved with the features of the present set of claims. The dependent claims specify preferred embodiments. In the process it is to be kept in mind that the named area specifications are to be understood without exception as including the respective limits.

Accordingly a wound care article is provided exhibiting a quantity of super-absorbent polymers. The wound care article is characterized in that it exhibits at least one agent which can restrict the bleeding or the tendency to bleed.

In the case of said polymers it is a matter of synthetic materials which are able to absorb a multiple of their inherent weight—up to 1000 times. These synthetic substances can be present in the form of granulate material, powder, fill, pellets, foam, in the form of fibers, of a fiber fabric, fiber non-woven fabric or non-woven fabric and/or a fiber wadding.

In the process, chemically it is as a rule a matter of a copolymer made of acrylic acid (propenoic acid, C₃H₄O₂) and sodium acrylate (sodium salt of acrylic acid, NaC₃H₃O₂), wherein the relationship of the two monomers to one another can vary. Additionally often a so-called core-cross-linker (CXL) is added to the monomer solution, which links the formed long-chain polymer molecules in places to one another through chemical bridges. Through these bridges the polymer becomes water-insoluble. In the case of the penetration of water or aqueous salt solutions into the polymer particle it swells up and tights at the molecular level of this network—the water can no longer escape without help.

Super-absorbent polymers are used for the most part in baby diapers, however also in women's hygiene products, for incontinence care or for the treatment of non-bleeding, but chronically exudating wounds.

The absorptive capacity for fluids is strongly dependent on the composition of the fluid. The best absorptive capacity exists with respect to distilled water. However a clearly reduced absorptive capacity is already to be observed compared to urine with a salt content of about 2 percent by weight. Blood, which exhibits a solids content (Hematocrit) of about 40% and beyond that a high content in dissolved salts in the serum, is absorbed even more poorly.

Preferably provision is to be made in the process that the agent is at least one chemically and/or pharmacologically and/or physiologically acting active ingredient or active ingredient complex. Likewise the agent can be at least one physically acting active element.

The terms “chemically and/or pharmacologically and/or physiologically acting active ingredient or active ingredient complex” and “physically acting active element” will be explained later.

Preferably the chemically and/or pharmacologically and/or physiologically acting active ingredient or active ingredient complex is at least one substance or a composition selected from the group containing:
Tranexamic acid
Hemostatic cotton, exhibiting oxidized cellulose and/or iron chloride
Hemostatic means from dentistry
Gelatins, preferably of bovine, equine and/or porcine origin, especially preferably of recombinant origin
Herbal gelatins
Preparations based on oligosaccharides or polysaccharides, such as starch, potato starch
or potato starch, preferably oligosaccaride or polysaccharide of glucose
oxidized regenerated cellulose
substituted cellulose, preferably carboxylated cellulose, especially preferably carboxymethyl cellulose
Fibrin agglutinating substances, if necessary of human origin
Blood coagulation factors I through XII, in particular VII through IX (if necessary also activated)
Inhibitors of the inhibitors of the blood coagulation factors
Plasma protein, human with Factor VIII inhibitor
Collagen, preferably of bovine, porcine and/or equine origin, especially preferably of recombinant origin
Herbal collagen, for example from sea weed or acacia or wheat proteins
Extensin, an herbal glycoprotein with high hydroxyproline content
Thrombocyte aggregation promoters
Antagonists of the inhibitors of Cyclooxygenase 1 and 2
Adenosine diphosphate
Lipoxin
Leukotriene
Inhibitors of the prostaglandins
Derivatives of arachidonic acid
Thromboxanes such as TXA2 and TXB2, PGH2
Epinephrine
Fibrin fission products
Antifibrinolytic agents
Antagonists for the Tissue Factor Pathway Inhibitor (TFPI),
Antagonists for Thrombomodulin together, with Protein C and S,
Antagonists for Antithrombin III
Antagonists for Heparin or Heparan sulphates
Antagonists for a2-Macroglobulin
Antagonists for C1 inhibitors
Thrombin, prothrombin and prothrombin activator
Platelet reactive coagulation factors
Thrombomodulin
Tretinoin
Thrombin-activated fibrinolysis inhibitor (TAFI)
Chemically defined or enzymatic antihemorrhagics, preferably antifibrinolytic agents, e.g. based on amino acids such as tranexamic acid, 4-(Aminomethyl)benzoic acid, phytomenadion (Vit.K₁), terlipressin acetate,
Protease inhibitors such as aprotinin solutions
Vitamins of the K group
Coagulable fibrinogen
Eptacog alfa (activated)
Nonacog alfa
human thrombin
calcium ion donors, such as calcium chloride solution
Fibronectin
plasma fibronectin
basic bismuth gallate
Tannin
Metallic platelets or plates, which exert a cooling, compressing and/or astringent effect
Astringent acting means
Alum salts, such as potassium aluminium sulfate and aluminium ammonium sulfate
Sympathomimetic drugs, such as xylometazoline, oxymetazoline, catecholamines such as adrenalin and noradrenalin
Methylxanthines such as caffeine and theine
Nicotine
Chitosan
Sodium alginate
Chitosan niacinamide ascorbate
Monsel's solution
iron sulfate

Additionally the chemically and/or pharmacologically and/or physiologically acting active ingredient or active ingredient complex is at least one extract from a medicinal plant, selected from the group containing:
Plants of the genuses Calendula, Echinacea, Pelargonium (preferably P. sidoides)
Common horsetail (Equisetum arvense)
Acanthus (Acanthus mollis)
Columbine (Aquiiegia vulgaris)
Arnica (Arnica Montana)
Brooklime (Veronica beccabunga)
Barbarakraut (Barbarea vulgaris)
Comphrey (Symphytum officinaie)
Blessed thistle (Cnicus benedictus)
Fluxweed (Descurainia Sophia)
Betony (Szachys officinalis)
Burnet saxifrage (Pimpinella major)
Tormentil (Potentilla erecta)
Common self-heal (Prunella vulgaris)
Figwort (Scrophularia nodosa)
Artemisia
Ivy (Hedera helix)
Sedum (Sedum telephium)
Cinquefoil (Potentilla reptans)
Wall germander (Teucrium chamaedrys)
Daisy (Bellis perennis)
Silverweed (Potentilla anserine)
Loosestrife (Lysimachia vulgaris)
Goldenrod (Solidago virguarea)
Ground ivy (Glechoma hederaceum)
Common bugle (Ajuga reptans)
Hawkweed (Hieracium pilosella)
Weeping birch (Betula pendula)
Houseleek (Sempervivum tectorum)
Blueberry, here: juice (Vaccinium myrtiilus)
Hart's tongue (Phyilitis scolopendrium)
Shepherd's purse (Capsella bursa-pastoris)
Coltsfoot (Tussilago farfara)
Briar rose, “Dog rose” (Rosa canina)
Chamomille (Matricaria chamomilla)
Catnip (Nepeta cataria)
Catsfoot (Antennaria dioica)
Common clubmoss (Lycopodium clavatum)
Robin-run-in-the-hedge (Galium aparine)
Burr (Arctium lappa)
Garlic germander (Teucrium scordium) in powder form (scentless)
Garlic mustard (Alliaria petiolata)
King fern (Osmunda regalis)
Groundsel (Senecio vulgaris)
Cleaver (Galium verum)
Lavender (Lavendula officinalis)
Common scurvy grass (Cochlearia officinalis)
Myrtle (Myrtus communis)
Kidneywort (Timbicilus pendulinus)
Wood avens (Geum urbanum)
Agrimony (Hgrimonia eupatoria)
Birthwort (Aristolochia clematitis)
Butterbur (Petasites hybridus)
Spindle tree (Euonymus europaea)
Moneywort (Lysimachia nummularia)
Devil's gut (Cuscuta epithymum)
Nipplewort (Lapsana communis)
Rampion (Camanula rapunculus)
Horse chestnut (Aesculus hippocastanum)
Herb Robert (Geranium robertianum)
Sage (Salvia officinalis)
Wood sanicle (Sanicula europaee)
Yarrow (Achillea millefoiium)
Bistort (Polygonum bistorta)
Black alder (Alnus glutinosa)
Black poplar (Populus nigra)
Carline thistle (Carlina acaulis)
Gooseberry (Ribes uva-crispa)
Melilot (Melilotus officinalis)
Wormseed (Artemisia maritima)
Dead nettle (Lamium album)
Common centaury (Centarium erythraea)
Devil's bit scabious (Succisa pratensis)
Thyme (Thymis vulgaris)
St. John's wort (iipericum perforatum)
Knotweed (Polygonum aviculare)
Chickweed (Stellaria media)
Juniper (Juniperis comxunis)
Common Gypsyweed (Veronica officinalis)
Common honeysuckle (Lonicera periclymenum)
Sweet woodruff (Galium odoratum)
Hemp agrimony (Eupatorium cannabinum)
Arsesmart (Polygonum dydropiper)
Firewood (Epilobium augustifolium)
Absinthe (Artemisia absinthium)
Great burnet (Sanguisorba officinalis)
Salad burnet (Sanguisorba minor)
Kidney vetch (Anthyllis vulneraria)
Male fern (Dryopteris filix-mas)
Chrysanthemum (Tanaceum balsamica)
Pear tree (Pyrus communis)
Boy's love (Artemisia abrotanum)
French rose (Rosa gallica)
Collard (Brassica oleracea)
Leek (Allium porrum)
Madonna lily (Lilium candidum)
Walnut tree (Juglans regia)
Onion (Allium cepa)
Benzoin (Styrax tonkinensis)
Cola tree (Cola acuminate)

The chemically and/or pharmacologically and/or physiologically acting active ingredient or active ingredient complex can be present in dry form and e.g. be integrated in a non-woven fabric which is the component of the inventive wound care article. However, it can also be present in dissolved form. For this purpose a solution or a gel can be produced which contains the said acting active ingredient or active ingredient complex. Preferably n the process an aqueous and/or alcoholic solvent is used. For example a wound dressing which is a component of the inventive wound care article can be soaked with this solution or gel. Such an article can be present ready-made or can also be present in the form of a kit consisting for example of a wound dressing and a gel containing said chemically and/or pharmacologically and/or physiologically acting active ingredient or active ingredient complex.

Basically it appears at first contradictory to use super-absorbent polymers in a wound care article which is intended to be used for hemostasis. However in such cases it is supposed to be the objective of the treatment to prevent the further leakage of fluids, in particular of blood, from the wound and, not for example to allow fluids to leak out and to absorb them.

This is in particular of significance because wound dressings containing super-absorbers occasionally can absorb very large quantities of fluids. Thus the product Sorbion Sachet S 20-10 of the manufacturer Sorbion Deutschland absorbs up to 400 ml of fluid.

The approach followed by the inventors to use the inventive wound care article therefore prevents a direct contact between an acute, profusely bleeding wound and the wound care article. In the process in particular two types of usage are provided:

In the case of chronic wounds, which if applicable exhibit a slight bleeding, as occurring e.g. in the case of Ulcus cruris, decubitus or similar ailments, provision is made that the wound dressing is brought into direct contact with the wound. Here the danger of a critical loss of blood is not to be feared. On the one hand here the absorption of exudates as well as the restriction of further bleeding or of the tendency to bleed through the relevant agents are the main priorities. Initial additional extravasating blood or at least its serous parts should here moreover be absorbed by the superabsorber of the wound dressing.

In particular in the case of chronic wounds the wound care article however can also be configured as a compression dressing and/or can be integrated in one. This is in particular indicated in the case of such wounds which have edematous character, thus e.g. Ulcus cruris. Here the compression improves the state of the vessel walls, i.e. the endothelial cell structure is reconstructed and the interstitial spaces through which fluid can extravasate into the tissue, is prevented. In addition to this comes the fact that the functionality of the venous valves is restored again through the compression and thus the venostasis is reduced. A combination of such compression with an inventive absorbing wound care article which exhibits a chemically and/or pharmacologically and/or physiologically acting active ingredient or active ingredient complex is very promising. The wound care article in the process absorbs primary exudates, however also blood which initially extravasates from the chronic wound in spite of the administered hemostatic active ingredient or active ingredient complex.

In the case of acute bleeding wounds, such as can e.g. be evoked by an acute trauma, on the other hand provision can be made that
a) the wound care article is configured as a tourniquet or compression dressing and/or can be integrated into one, and/or
b) the wound care article is arranged downstream of an arterial Spanish tourniquet.

In these cases the brunt of the hemostasis lies with the tourniquet or compression dressing or with the Spanish tourniquet. In both cases it is a matter of physically acting active elements in the above sense. The task of absorbing initial additional extravasating blood through the superabsorber resides in the process with the wound care article.

Further physically acting active elements in terms of the invention are e.g. active elements which introduce cold into the wound, e.g. the so-called ice packs, but also transient substances which produce evaporative cooling, thus e.g. ethanol, isopropanol or other organic solvents.

In connection with the above stated a special significance is attributed to the super-absorbent polymers that are used.

Since it is true that these absorb fluids, but not the cellular components of the blood, at the outside of the inventive wound care product there is an agglomeration of cellular components.

The wound care article can preferably exhibit an essentially flat material segment made of absorption material, which consists of an absorbent fleece with super-absorbent polymers distributed within. Said super-absorbent polymers can be present in the form of particles, but also in the form of fibers or of a fiber composite, in the form of a foam or the like. Further the wound care article can exhibit a fluid-permeable sleeve which encloses the material segment and forms a barrier against solid excretions and makes possible the passage of other extravasated substances to the material segment made of absorption material arranged within the sleeve. The sleeve is preferably closed at least partially by a seam.

Especially preferably the material segment exhibits in top view on its flat side an area (F1) which in its non-wetted state is 1% to 90% smaller than the area (F2) of the sleeve when it is laid flat and is freely moveable or fixed in the sleeve, wherein the sleeve or one of its layers exhibits pores over its entire area which are each smaller than the unwetted super-absorbent polymers.

Especially preferably provision is made that the sleeve in top view on its flat side exhibits a rotary protrusion protruding beyond the seam and the absorption body is free from hard, sharp edges and corners.

The pores or meshes of the sleeve are preferably 0.01 mm to 2.0 mm, preferably 0.20 mm to 0.50 mm large. In addition preferably provision can be made that the pores or meshes are bordered by thread or fiber segments which in the section through the sleeve are somewhat arch-shaped and point with their arch vertices outward.

The inside layer of the absorption body exhibits preferably an area-specific weight of 100 g/m²-600 g/m², wherein the amount of the super-absorbent polymers distributed within lies within the range of 25-100 percent by weight. Especially preferably the amount of the super-absorbent polymers lies within the range of 15-100 percent by weight.

The wrapping is preferably formed of woven or fleece-like composed synthetic fibers, such as polypropylene or polyethylene fibers. It consists preferably of a fabric or a fleece, which exhibits an area-specific weight in the range of 10-1000 g/m², especially preferably 10-100 g/m².

The connection between the wrapping and a material segment arranged within is preferably accomplished by splices, welds, seams, lockstitch seams, bonding points, embossing or by thermo-mechanical bonds.

In the case of said bonding points it is a matter of press points which with the help of a press are applied to the wound care article and which provide for a thermal and/or physical connection of the various layers of the wound care article. The bonding points as a rule are present in a regular pattern.

In the process in particular hydrophobic materials such as polyurethane, polyethylene or Teflon are especially preferred for the wrapping, since—in the case of corresponding design—the fluid passage can be modulated with their help. Thus e.g. a delayed fluid passage is definitely practical in the case of the use of super-absorbent polymers in order to prevent a too rapid drainage of the wound. Moreover most hydrophobic materials exhibit a smooth and inert surface, as a result of which bonding with the wound are prevented.

This modulation of the fluid passage can be ensured by means of special measures. This can for example be perforations or pores introduced to the sleeve or lamination. Likewise provision can be made that the above named measures for planar or point connection between the sleeve and the flat material segment gets the double role of bringing about a fluid-permeability of the sleeve or lamination.

In this connection in particular thought is being given to the aforementioned bonding points. These exhibit recesses of the sleeve material upon whose base (i.e. in their region contacting the flat material segment or in the same penetrating region) the material is stretched and therefore exhibits micro-perforations which make fluid passage possible. In this embodiment the bonding points thus in addition to the object of joining the different layers of the wound care article to each other also have the object of ensuring the passage or entry of fluid. In addition comes the fact that the bonding points exhibit an effect that enlarges the surface, which further increases the suction efficiency.

Likewise thought is being given to the aforementioned lockstitch seams. These too can ensure a fluid passage through the hydrophobic sleeve material.

Wound dressings of the named type are disclosed for example in WO03094813, WO2007051500 and WO0152780 of the applicant of the present invention. The disclosure content of the named publications is added in its entirety to the disclosure content of this publication.

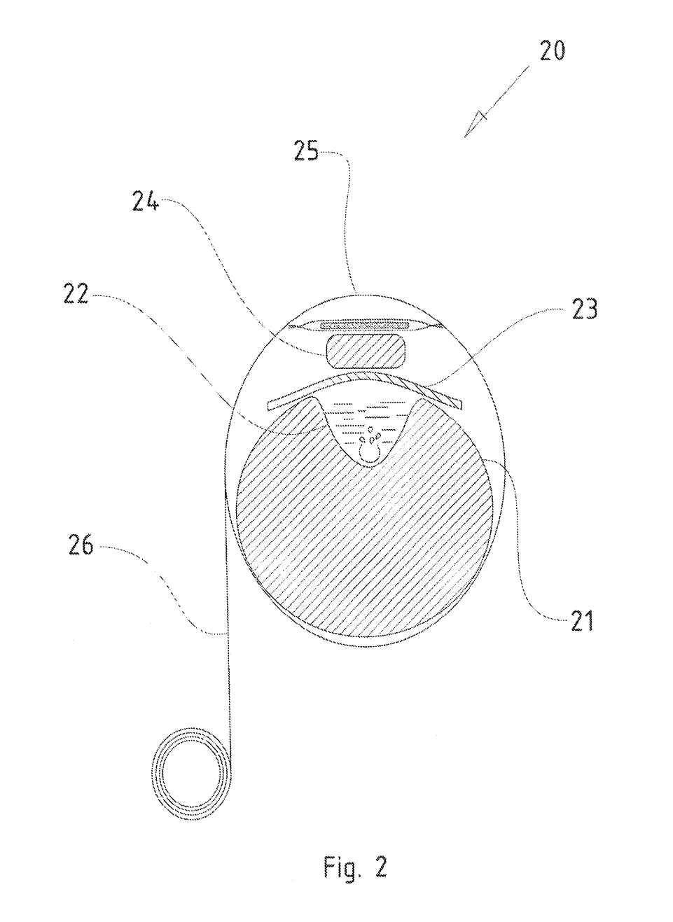

Said wound dressing contains at least one chemically and/or pharmacologically and/or physiologically acting active ingredient or active ingredient complex and is suitable in particular for hemostasis in the case of chronic wounds which if applicable exhibit slight bleeding or in the case of acutely bleeding wounds as or in combination with a tourniquet or compression dressing (FIG. 2) or downstream of an arterial Spanish tourniquet.

Said at least one chemically and/or pharmacologically and/or physiologically acting active ingredient or active ingredient complex is in the process preferably arranged in the region of the wound dressing facing the wound.

In the embodiment with a tourniquet or compression dressing moreover it is advantageous that any fluid seeping through which is absorbed by the super-absorbent polymers lead to a swelling of the latter, as a result of which in turn the physical pressure exerted on the wound is increased.

In addition the wound care article can exhibit a primary, non-absorbing or only marginally absorbing wound dressing which exhibits at least one chemically and/or pharmacologically and/or physiologically acting active ingredient or active ingredient complex, as well as a secondary wound dressing arranged peripheral from this primary wound dressing, which contains super-absorbing polymers. In the case of said secondary wound dressing it can for example be an essentially flat material segment made of absorption material which consists of an absorbent fleece with super-absorbent polymers distributed within. While the primary wound dressing has the task of staunching the bleeding, the secondary wound dressing has the task of absorbing further blood extravasating from the wound by the superabsorber. Said wound care article can additionally exhibit a separating layer between the primary and secondary wound dressing, which acts as a diffusion barrier and prevents the superabsorber arranged in the secondary wound dressing from exerting a suction effect on the wound and in this way forcing the bleeding. Said diffusion barrier can be embodied in such a way that blood or serum fluid only seeps through the barrier from a specified hydrostatic pressure and is then absorbed by the secondary wound dressing. Likewise the diffusion barrier can exhibit pores whose diameter is lesser than that of the blood cells. Thus erythrocytes exhibit e.g. diameters of between 6 and 10 μm, while thrombocytes exhibit diameters between 1.5 and 3.0 μm. The pores in said diffusion barrier could therefore exhibit e.g. diameters between 1 and 8 μm.

Under circumstances such a pore size is however not even necessary. Erythrocytes tend under the influence of blood coagulation factors and thrombocytes to an aggregation. Therefore it is to be expected that the erythrocytes in the region of the wound dressing form aggregations which exhibit significantly greater diameters than the erythrocytes themselves and in this way also a diffusion barrier with significantly higher pore sizes can actively prevent the passage of cellular components.

In addition comes the fact that the wound dressing preferably absorbs fluid and thus leads to a fluid depletion in the region of the wound. This in turn increases the aggregation tendency of the erythrocytes, because the hematocrit in the environment increases thus.

The said diffusion barrier can in the process be configured in the form of a sleeve which is arranged around the secondary wound dressing. This embodiment corresponds for example in one embodiment to the product Sorbion sachet that has already been described.

Here too the sleeve can exhibit pores of the said size, so that the secondary wound dressing only absorbs the fluid components of the blood, but not the cellular components, which are concentrated before the sleeve.

Likewise the diffusion barrier can exhibit strongly hydrophobic properties or can be embodied in such a way that a fluid continuum between the wound and the superabsorber is prevented for example by air pockets. Said features can also be combined with one another.

For this purpose the diffusion barrier can e.g. be embodied as a semi-permeable foil or membrane made of the hydrophobic material PTFE (polytetrafluorethylene). Likewise the diffusion barrier can be embodied as a foam mat made of foam exhibiting hydrophobic air pockets.

Additionally the diffusion barrier can be a three-dimensional perforated foil, if applicable applied in multiple layers, as is known for example from DE 102006017194 of the applicant of the present invention. Here too a fluid continuum between the wound and the superabsorber is suppressed prevented by air pockets.

Additionally said diffusion barrier can exhibit at least one layer made of oxidized cellulose. Said layer exhibits on the one hand hemostatic properties, on the other hand the pore size can be selected in such a way that the blood cells cannot pass this layer.

Additionally said diffusion barrier can be an if applicable applied in multiple layers, three-dimensional perforated foil, as is known for example from DE 102006017194 of the applicant of the present invention, whose disclosure content is to be completely included here. Here too a fluid continuum between the wound and the superabsorber is suppressed prevented by air pockets.

Also in this embodiment it is advantageous that any fluid seeping through which is absorbed by the super-absorbent polymers leads to a swelling of the latter, as a result of which in turn the physical pressure exerted on the wound is increased.

Moreover such a wound care article can additionally exhibit a pressurized cushion. The pressurized cushion can for example be a fluid-proof packed cushion made of cellulose, likewise however it can for example be a handle or a spherical object.

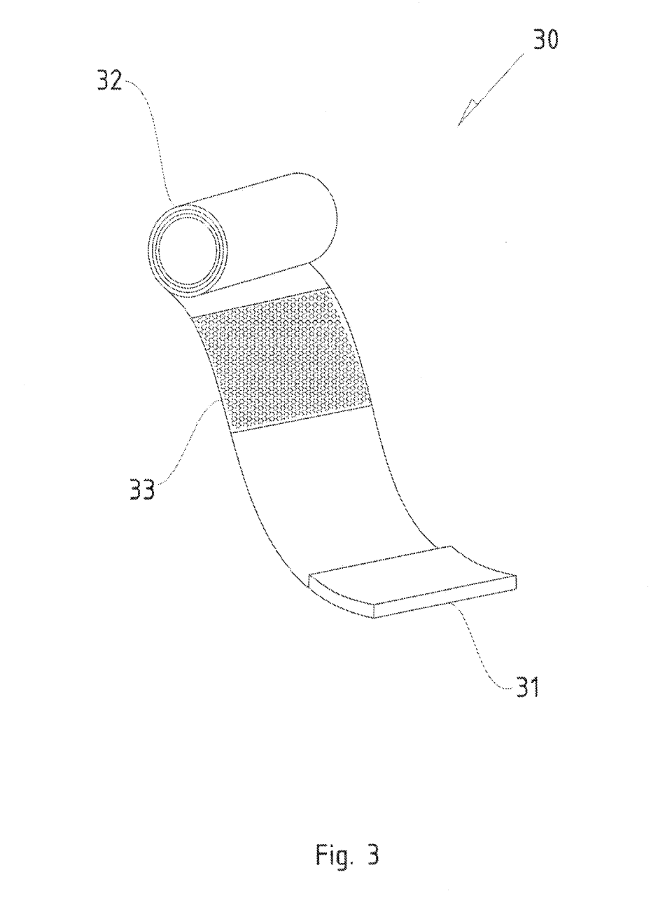

Also the wound care article can be embodied in the form of a wound dressing packet, exhibiting a primary wound dressing with at least one chemically and/or pharmacologically and/or physiologically acting active ingredient or active ingredient complex as well as a compress segment arranged on the wound dressing which exhibits at least in segments super-absorbent polymers (FIG. 3). Moreover such a wound care article can additionally exhibit a pressurized cushion. The pressurized cushion can for example be a fluid-proof packed cushion made of cellulose; likewise however it can for example be a handle or a spherical object.

Also in this embodiment it is advantageous that any fluid seeping through which is absorbed by the super-absorbent polymers leads to a swelling of the latter, as a result of which in turn the physical pressure exerted on the wound is increased.

In addition the wound care article can be configured in the form of a material segment with a longitudinal extension exhibiting absorption material, wherein the material segment exhibits if applicable elastically deformable properties, and wherein the material segment exhibits super-absorbent polymers as well as if applicable at least one chemically and/or pharmacologically and/or physiologically acting active ingredient or active ingredient complex.

This material segment can for example be arranged in the form of a tube around a limb which exhibits a profusely bleeding wound. Due to the elastic properties in the process a pressure can be applied to the wound which leads to a physical hemostasis. Moreover if applicable existing chemically and/or pharmacologically and/or physiologically acting active ingredients or active ingredient complexes can contribute to the hemostasis. Any additional extravasating blood will then be absorbed by the super-absorbent polymers.

Such a wound care article can also be configured as a tear-off item. For this purpose the material is for example arranged around a coil or roll and can in cases of need be torn off in the appropriate length. For this purpose the material can if applicable exhibit transversal perforations in order to facilitate the tearing off.

Such an article is especially well suited to wound care in emergency medicine, in particular in the case of injuries due to accidents, such as industrial and traffic accidents or in the case of shot or explosion injuries. Thus such an article configured as a tear-off item as described above can be a component of an emergency or military medical kit, thus e.g. of a first-aid kit, an emergency kit, equipment for emergency services or an ambulance.

Preferably in this connection provision is moreover made that the wound care article in addition exhibits at least one nutritive, at least one disinfecting or decontaminating and/or at least one protease inhibiting acting active ingredient or active ingredient complex.

In the case of the disinfecting acting active ingredient or active ingredient complex it can for example be a composite of at least one vitamin or vitamin derivative, a metallic ion as well as a detergent. Likewise it can be a BLIS (bacteriocin like inhibitory substance) or a coated magnetic particle.

In the case of the nutritive acting active ingredient or active ingredient complex it can be a composition containing at least the components of an enteral and/or parenteral dietary agent. Likewise it can be at least one active element selected from the group containing insulin, recombinant insulin, proinsulin, an insulin-like growth factor (IGF), an insulin mimetic and/or a diabetic-specific, non-glucose or saccharose-based energy source.

In the case of the protease inhibiting acting active ingredient or active ingredient complex it can be at least one active element selected from the group containing protease inhibitors, super-absorbent polymers, chelating agents for bivalent cations, collagen, coated magnetic particles, acids, buffers, non-pathogenic acid producing microorganisms, probiotics and/or symbiotics.

Additional combinations and backgrounds for the nutritive disinfecting, decontaminating or protease inhibiting acting active ingredients and/or active ingredient complexes are described in DE 10200703 0931 of the applicant of the present application, to whose contents complete reference is made here. In DE 10200703 0931 other nutritive, disinfecting, decontaminating and/or protease inhibiting acting active ingredients and/or ingredient complexes are described which are likewise intended to be considered disclosed in this application.

In addition the inventive wound care article can also be incorporated in a wound care system for wound drainage using a vacuum. Such systems are for example disclosed in the publications DE202004017052, WO2006048246 and DE202004018245 of the applicant of the present invention, whose disclosure content is to be considered as belonging to the present invention.

From the first mentioned publication a device for the treatment of wounds using a vacuum is known, exhibiting a gas-tight wound dressing element which in the state of being placed on the body of the patient forms a space remaining between the respective wound and the wound dressing element, and at least one connector point, which is in contact with the space and via which the air in the space can be evacuated, wherein the wound dressing element is supported by at least one extensive wound care article absorbing the wound secretion, whose volume increases in the course of the absorption process, so that the absorbed wound secretions within the wound care article and with it remains underneath the wound dressing element until removal of the wound care article from the body of the patient, the wound care article is at least one layer with super-absorbent fortified textile segment, which is enclosed by a fluid-permeable sleeve and the layer in top view on its flat side has an area which is 3% to 90% smaller than that of the sleeve, so that the wound care article can, in the proximity of its entire filling capacity approximate in cross-section a circular shape.

From the second mentioned publication a multiple component bandage for the treatment of wounds of the human or animal body using a vacuum is known, exhibiting:

a wound dressing element for application to skin and mucous membrane surfaces, at least one connector point which is in contact with the space and via which the air in the space can be evacuated, wherein said multiple component bandage exhibits super-absorbent polymers, wherein the absorbed wound secretions remain bound on polymers in the wound space until removal from the wound space, wherein the polymers through their binding capacity support reciprocal synergies with the sub-atmospheric pressures.

From the latter mentioned publication a drainage device for the treatment of wounds using a vacuum is known, exhibiting a gas-tight wound dressing element consisting of foil-like material which in the state of being placed on the body of the patient is adhesively attached on the skin surface around the wound region and which forms a sealed up space remaining between the respective wound and the wound dressing element, at least one drainage hose which can be inserted into the space, via which the substances in the space can be evacuated and at least one wound care article arranged within the space which absorbs the wound secretions, which exhibits at least one layer of a textile segment fortified with superabsorbers, which is enclosed by a fluid-permeable sleeve, wherein the absorbed wound secretions remain within the wound care article and with this below the wound dressing element until removal of the wound care article from the body of the patient and wherein the wound dressing element exhibits a gas-tight closable treatment opening through which the wound care article can be inserted into and removed from the space.

The inventive wound care article can moreover exhibit a form adapted to anatomical circumstances. For this purpose it can for example be configured in the form of a cuff; which can be put over an arm or a leg or a joint, or in the form of a bandage adapted to the heel, the elbow joint or the like.

The inventive wound care article can moreover be configured in such a way that it is suitable for placing around a line inserted by surgery. For this purpose the wound care article can for example exhibit at least one slit which makes it possible to place the bandage on the body of a patient around a line (e.g. a drainage line or a catheter), wherein the wound care article has a second, likewise flat wound care article assigned to it which is at a distance from the first wound care article, wherein the distance is bridged by a connection strip or web. Such a wound care article is for example known from DE202006005966 of the applicant of the present invention, whose contents are intended to be completely added to the disclosure content of the present description.

In accordance with the invention moreover provision is made for the use of a wound care article as described above for traumatically produced wounds. Here in particular thought is being given to wounds as they occur in street traffic, at sporting events, in the case of physical labor, at accidents, through firearms or due to assault offenses. All of these wounds as a rule are characterized by profuse bleeding which is frequently evoked by injured arteries. In principle in the process especially preferably the mentioned physically acting active elements are possible.

Likewise in accordance with the invention provision is made for the use of a wound care article as described above for military purposes. Here in particular the care of field injuries is the main priority, as or example evoked by firearms, explosives or the like. Here too especially preferably the mentioned physically acting active elements are possible.

In this connection also the disinfecting and decontaminating properties of the super-absorbent polymers are of importance. Thus super-absorbent polymers can, on the basis of their absorptive capacity absorb and immobilize germs as well as also poisonous substances and radiating particles. For this reason the inventive wound care article is also suitable for the treatment of injuries caused by radiation contamination, burns, chemical burns, poisoning or the influence of biological weapons. The wound care article can therefore for example be used in the first care of atomic, biological or chemically contaminated soldiers. After use the wound care article must be properly disposed of.

In addition in accordance with the invention provision is made for the use of a wound care article as described above for the treatment of wounds which, due to a hemophilia disorder or due to a systemically conditioned or pharmacologically evoked coagulation deficit exhibit a deteriorated healing process.

Hemophilia is an inherited disease in which the blood coagulation is disturbed. The blood from wounds does not coagulate or coagulates only slowly. Frequently there is also spontaneous bleeding which occur without visible wounds. Hemophilia occurs mainly in men, since it is as a rule caused by an X chromosomal recessive gene.

A pathological coagulation deficit can for example be evoked by a liver disease, such as e.g. cirrhosis of the liver.

A systemically caused coagulation deficit can for example also be inherited, thus e.g. the so-called APC resistance, but can also be caused also by a generally poor overall constitution of the patient. The case configuration can also be counted as such, in which case the patient has previously donated blood or thrombocytes or has donated coagulation factors.

A pharmacologically evoked coagulation deficit can for example occur within the framework of a heart attack, a stroke or a thrombolysis therapy or prophylaxis. Causes for this are coagulation inhibitors administered to the patient, such as e.g. Marcumar or so-called blood thinners, such as e.g. acetylsalicylic acid. Pharmacologically evoked coagulation deficits after chemotherapy and/or radiation therapy within the framework of cancer treatment likewise occur.

The use of the inventive wound care article can be of great advantage for all of these indications. Basically in the process especially preferably the mentioned chemically and/or pharmacologically and/or physiologically acting active ingredients or active ingredient complexes are possible.

In addition in accordance with the invention provision is made for the use of a wound care article as described above for operative or post-operative care. Also in the case of such indications a rapid hemostasis is frequently top priority. Basically the mentioned chemically and/or pharmacologically and/or physiologically acting active ingredients or active ingredient complexes as well as also the mentioned physically acting active elements are possible here.

### DEFINITIONS

The term “wound care article” should in the following in particular denote a wound dressing, preferably a planar wound dressing or a wound care cloth. Said wound dressing can in the process be configured absorbent as well as also non-absorbent or only marginally absorbent. In particular the term “wound care article” can also be understood as an ensemble of different products which are arranged in a given arrangement on the wound to be treated. This ensemble can form a physical unit, by combining the different products in a common sleeve or—if applicable without sleeve—be adhesively joined to one another. The ensemble can however also be present in the form of a kit, in which the different products are arranged with the help of a compress in the given arrangement on the wound to be treated.

The term “exudate” denotes a wound fluid derived from blood plasma via the inflammatory processes of wound edema. Just as blood is responsible for the transport of nutrients and other messengers and with this for the care of different parts of the body, the exudate serves in quite similar fashion the purpose of the care of the wound bed and the healing processes running therein. In order to do justice to the vast number of functions it contains a broad spectrum of components, from which a specific weight results which lies just above that of water. In this it also differs from transsudate which is derived from non-inflammatory processes and which exhibits a significantly lower specific weight with a lower cell and protein content. Along with the provision of nutrients for the fibroblasts and epithelial cells, the exudate coordinates the different processes of wound healing chronologically and spatially through its high content in growth factors and cytokines These are formed above all by thrombocytes, keratinocytes, macrophages and fibroblasts. They influence the motility, migration and proliferation of the different cells involved in the healing of wounds. Thus the migration of cells into the wound base is likewise promoted as the care of the newly formed granulation tissue by angiogenesis. Also the wound cleansing procedure is supported by the exudate. It contains different serine, cysteine and aspartate proteases as well as matrix metalloproteases, which are strictly regulated in their activity break down irreversibly damaged tissue and hence prepare the wound bed for the subsequent phase of healing.

Components of the physiological exudate are in particular salts, glucose, cytokines and growth factors, plasma proteins, proteases (in particular matrix metalloproteases), granulocytes and macrophages.

The term “chemically and/or pharmacologically and/or physiologically acting active ingredient or active ingredient complex” should in the following be understood as those active ingredients or active ingredient complexes which are able to restrict bleeding or the tendency to bleed. The action path is here a chemical, pharmacological and/or physiological interaction with the wound milieu.

The term “active ingredient complex” is in the following to be understood not only as a complex in the chemical sense, but rather in particular as a composition of synergistic active ingredients evoking an effect.

The term “physically acting active element” is in the following to be understood as an active element which, by physical means, i.e. by the exertion of force, traction, cold and the like, is able to restrict bleeding or the tendency to bleed. Likewise such an active element can act by inherent physical properties or by physical interactions of an active ingredient or active ingredient complex, such as for example the exertion of capillary forces on the milieu.

The term “Spanish tourniquet” should in the following be understand as an emergency medical measure which is able to stop the arterial blood flow e.g. in a limb in order in this way to prevent an untenable loss of blood in a wound. Indications for such a Spanish tourniquet are as a rule traumatic effects which lead to the injury of at least an artery.

The term “downstream of an arterial Spanish tourniquet” should in the following be understood as a position on the body of a human or animal patient which in reference to the arterial blood flow seen from the heart is located distal of an arterial Spanish tourniquet.

The term “chronic wounds” should be understood as those wounds which cannot be attributed primarily to traumatic effects. While it is true that traumatic effects can be the original actuator of such a wound, but the chronic wound is characterized above all by a delayed wound healing. Chronic wounds frequently—if at all—exhibit only slight bleeding, but often on the other hand a profuse exudation.

The term “slight bleeding” should be understood as bleeding which is not of arterial origin, but rather if applicable of venous origin or interstitial or capillary origin and which in any event comes off so easily that it is not directly or indirectly life threatening.

The term “acutely bleeding wounds” should be understood as those wounds which lead to great loss of blood. As a rule arterial bleeding is responsible for this, which e.g. is caused by traumatic effects. Acutely bleeding wounds can under circumstances be directly or indirectly life threatening. For this reason in the case of acutely bleeding wounds hemostasis has a very high priority.

The term “tourniquet” should in the following be understood as the tourniquet known from emergency medicine, said tourniquet consisting of an object not too hard, non-absorbent object (Pressurized cushion) without sharp or hard edges, which is placed upon an already covered wound and with the help of a compress is attached with moderate traction. Through the exerted pressure the blood flow of the relevant body part is lessened and the traumatically opened blood vessels are closed again. Such a tourniquet is shown as a symbol in FIG. 2.

The term “not or only marginally absorbing wound dressing” should denote a wound dressing which exhibits a low absorptive capacity for fluids. Overall the absorptive capacity in the process should be less than 60% by weight, preferably less than 20% by weight of the dry weight of the wound dressing. The primary task of such a wound dressing is therefore not the absorption of blood or exudates, but rather the output of hemostatic agents in terms of the present invention.

The term “compression dressing” on the other hand is understood as a rule as a dressing which acts similar to a tourniquet, however without the mentioned pressurized cushion. The pressure or the compression on the wound is in this connection exerted exclusively by the compress. In this connection the compress material can be elastic.

In accordance with the present invention the inventive wound care article can be integrated into such a tourniquet or compression dressing or be configured itself as a tourniquet or compression dressing.

### DRAWINGS AND EXAMPLES

The present invention will be explained more closely by the figures and examples shown and discussed in the following. In the process it is to be kept in mind that the figures and examples only have describing character and are not intended to restrict the invention in any form whatsoever.

### Example 1

Substituted cellulose based on cellulose that has been chemically modified. The cellulose is an unbranched polymer of 1-4-β glycosidically linked glucose molecules with a chain length between one hundred and ten thousand monomers.

Preferably the substituted cellulose is cellulose ether, such as e.g. alkylated cellulose (e.g. methyl cellulose, ethyl cellulose, propyl cellulose), hydroxyalkylated cellulose (e.g. hydroxymethyl cellulose, hydroxyethyl cellulose, hydroxymethyl cellulose, hydroxypropyl-methyl cellulose) or carboxylated cellulose (e.g. carboxymethyl cellulose, carboxyethyl cellulose, carboxypropyl cellulose).

Preferably provision is made that the modified cellulose exhibits a substitution degree between and including 0.05 and including 3 alkyl, hydroxyalkyl or carboxyalkyl groups per glucose unit. In the process a substitution degree of 3 denotes three substituents per glucose unit, while a substitution degree of 0.05 denotes one substituent per 2000 glucose units.

Said chemically modified celluloses overflow upon contact with water successively into a gel-like state. In the process the gel forming property is dependent on the substitution degree of the cellulose, i.e. the higher the substitution degree, the earlier the gel formation occurs.

Substituted cellulose, in particular CMS, has a hemostatic effect, which on the one hand can be traced back to mechanical and on the other hand to blood physiological causes.

Thus CMS accelerates the agglutination of the blood platelets (thrombocytes) via fibrinogen or fibrin monomers and also accelerates the accumulation of the fibrin monomers to fibrin polymers. This means that CMS acts physically on a fibrinogen molecule or fibrin monomer. In addition CMS has a promotional effect on the cell adhesion and thus also promotes hemostasis. As a result of the property of CMC of forming a gel in the case of contact with moisture, moreover the bleeding is also mechanically stopped.

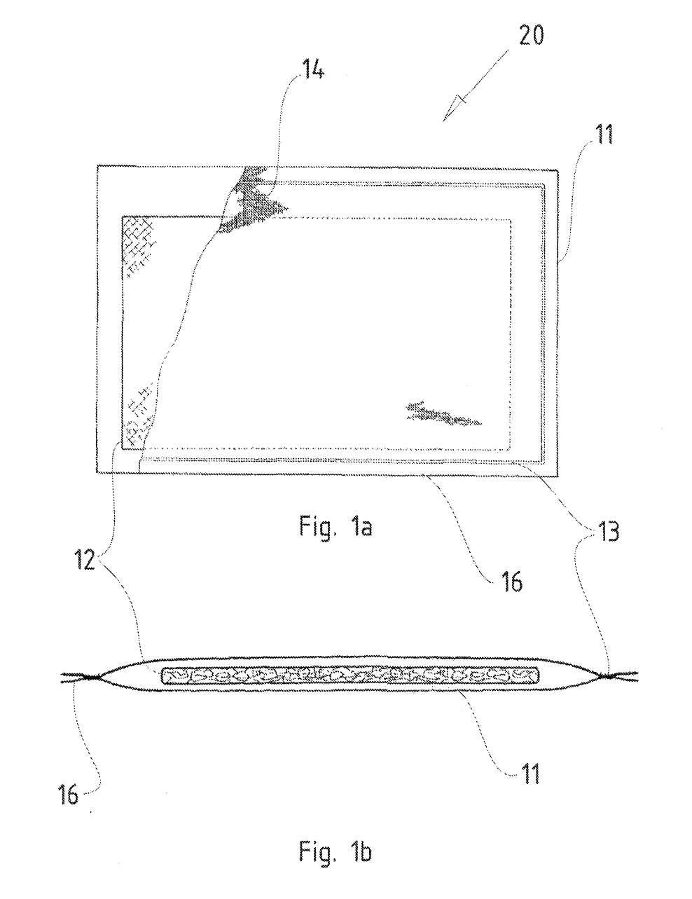

FIGS. 1a, 1b show an absorption body 10 which consists of a sleeve 11 and loose absorption material accommodated there in the form of a flat cellulose-like material segment 12.

The material segment exhibits a thickness of about 2 mm and exhibits a super-absorbent polymer. The polymer can be present in powder or granulate form, in the form of fibers, of wadding, a fleece or foam. Said powder can in the process be present as fill, as pellets or embedded in a non-woven fabric (“airlaid”). The sleeve 11 in accordance with FIG. 1 is made of two equal lateral walls connected to one another on their periphery via a weld seam 13. The area F1 of the material segment 12 in the process makes up about ⅔ of the area F2 of the sleeve 11. Through the differences between the areas F1 and F2 of the sleeve 11 and the absorption material 12 the absorptive capacity of the absorption material can develop completely. The sleeve 11 exhibits a close-meshed structure whose pores 14 in the present case are about 0.15 mm to 0.25 mm large and can for example consist of modified cellulose (preferably CMC).

One distinctive feature of the absorption body 10 is a rotary protrusion protruding over a seam 13 whose width lies between 1 mm and 5 mm. The task of the protrusion is to form a gentle buffer zone between the tissue of the patient and the seam 13 when the absorption body 10 is carelessly manipulated, i.e. when the absorption body touches the painful wound with its edge or corner.

The seam 13 is preferably constructed free of adhesives and binding agents, e.g. as a weld seam, preferably as an ultra-sound weld seam.

Provision is made for also using other, in particular square sleeves with the protrusion 5, in the case of said sleeves which the area Fl of the inner absorbent material segment is correspondingly smaller.

In addition the absorption body is preferably designed to be free of binding agents.

The described absorption body can in the case of chronic wounds which if applicable exhibit slight bleeding, such as e.g. occurs in the case of Ulcus cruris, decubitus or similar ailments, be brought into direct contact with the wound. Here the danger of a critical loss of blood is not to be feared. On the one hand the main priorities are the absorption of exudates as well as the restriction of further bleeding or the tendency to bleed through the relevant agents. Initial further extravasating blood should here moreover be absorbed by the super-absorber of the wound dressing. Said at least one chemically and/or pharmacologically and/or physiologically acting active ingredient or active ingredient complex can in the process be arranged in the region of the wound dressing facing the wound. In the case of acutely bleeding wounds, as e.g. evoked by an acute trauma, on the other hand provision can be made that
c) the absorption body is configured as a tourniquet or compression dressing and/or can be integrated in one, and/or
d) the absorption body is arranged downstream of an arterial Spanish tourniquet.

In these cases the brunt of the hemostasis lies with the tourniquet or compression dressing or with the Spanish tourniquet. In both cases it is a matter of physically acting active elements in the above sense. The object of absorbing initially additional extravasating blood by means of the superabsorber resides in the process with the wound care article.

FIG. 2 shows a tourniquet 20 which is placed around a body part 21 exhibiting a profusely bleeding wound 22. The tourniquet consists of a primary wound dressing 23 as well as not too hard, non-absorbent pressurized cushion 24 without sharp or hard edges, which is fastened with moderate traction with the help of a compress 26. In the case of the Pressurized cushion 24 it can be a matter e.g. of a fluid-proof packed cushion made of cellulose, likewise however it could for example be a handle or a spherical object. Through the exerted pressure the blood flow of the relevant body part is lessened and the traumatically opened blood vessels are closed again. In addition between the compress 26 and pressurized cushion 24 an inventive wound care article is arranged in the form of a secondary wound dressing 25 containing super-absorbent polymers. In the process the object of absorbing additional blood extravasating from the wound 22 in spite of the tourniquet by means of the superabsorber resides with the wound care article 25. In this embodiment it is moreover advantageous that any fluid seeping through which is absorbed by the super-absorbent polymers leads to a swelling of the latter, as a result of which in turn the physical pressure exerted on the wound is increased. The primary wound dressing 23 can moreover exhibit at least one chemically and/or pharmacologically and/or physiologically acting active ingredient or active ingredient complex.

FIG. 3 shows an inventive wound care article 30 which is structured similar to a conventional wound dressing packet, exhibiting a primary wound dressing 31 as well as a bandage compress 32 which is provided at least in segments with a super-absorbent polymer 33. The task of absorbing additional blood extravasating from the wound in spite of the tourniquet resides with the latter. The hemostatic effect of this wound care article can be evoked by at least one chemically and/or pharmacologically and/or physiologically acting active ingredient or active ingredient complex arranged in the primary wound dressing 31. As an alternative to or in addition to this the wound care article can be used as a tourniquet, in the process the primary wound dressing 31 acts as a pressurized cushion, or a separate pressurized cushion can be used. In this case thus the hemostatic agent is a physically acting active element. Also in this embodiment any fluid seeping through which is absorbed by the super-absorbent polymer leads to a swelling of the latter, as a result of which in turn the physical pressure exerted on the wound is increased.

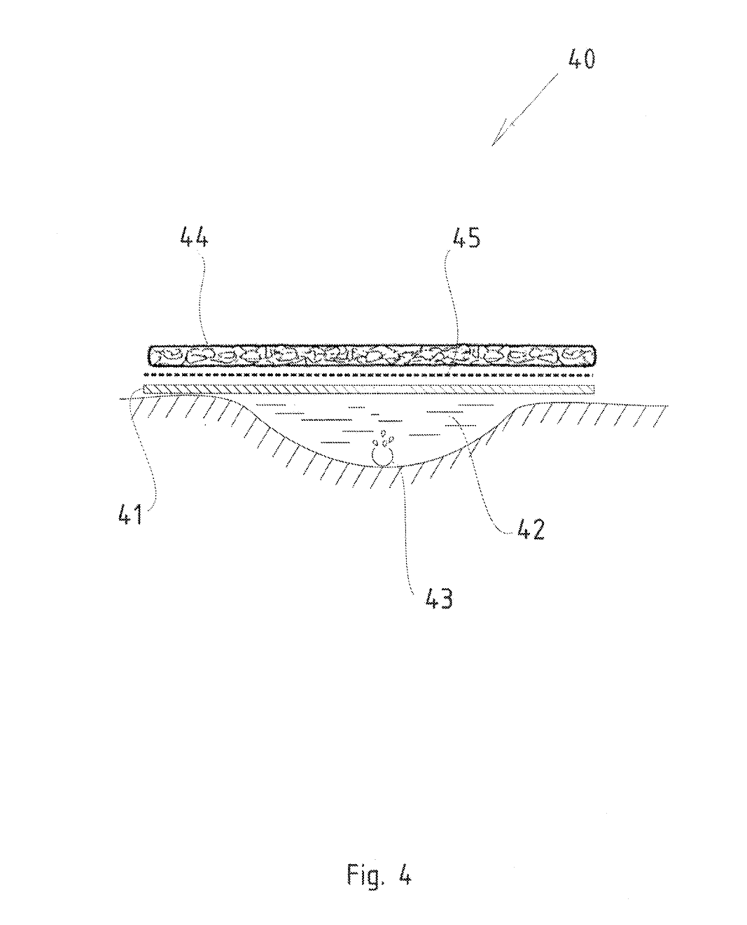

FIG. 4 likewise shows an inventive wound care article 40 consisting of a primary wound dressing 41 which exhibits at least one chemically and/or pharmacologically and/or physiologically acting active ingredient or active ingredient complex. This primary wound dressing 41 is placed directly on a wound 42 which exhibits a profusely bleeding injury of a vessel 43. The primary wound dressing does not act absorbent or acts only marginally absorbent. A secondary wound dressing 44 is arranged peripherally from this primary wound dressing, said secondary wound dressing corresponding e.g. to the wound dressing shown in FIG. 1 and containing super-absorbent polymers, but in contrast to the latter exhibiting no sleeve (but it can exhibit one). A diffusion barrier is arranged between the primary and secondary wound dressing, said diffusion barrier being embodied such that blood or serum fluid only passes through the barrier beginning at a specified hydrostatic pressure or that it exhibits pores whose diameter is smaller than that of the blood cells. This diffusion barrier can for example consist of modified cellulose (preferably CMC). In this way it is prevented that the super-absorber arranged in the secondary wound dressing exerts a suction effect on the wound and thus forces bleeding. A compress not shown in FIG. 4 can for example be arranged around the ensemble of primary wound dressing, diffusion barrier and secondary wound dressing in order to immobilize the three components on the spot. Likewise the three components can also be united in one compact, uniform product not shown in the figure. For this purpose they can for example be arranged in a common sleeve and/or be connected to one, e.g. by thermal welding, ultrasound seams, physiological adhesives such as starch or protein adhesives and the like. Such a product can for example be immobilized on the spot with plasters or a compress.

The shown diffusion barrier 45 can in some cases be dispensed with, e.g. if the super-absorber being used are embodied in such a way that they do not absorb any blood cells, but rather only blood serum fluid.

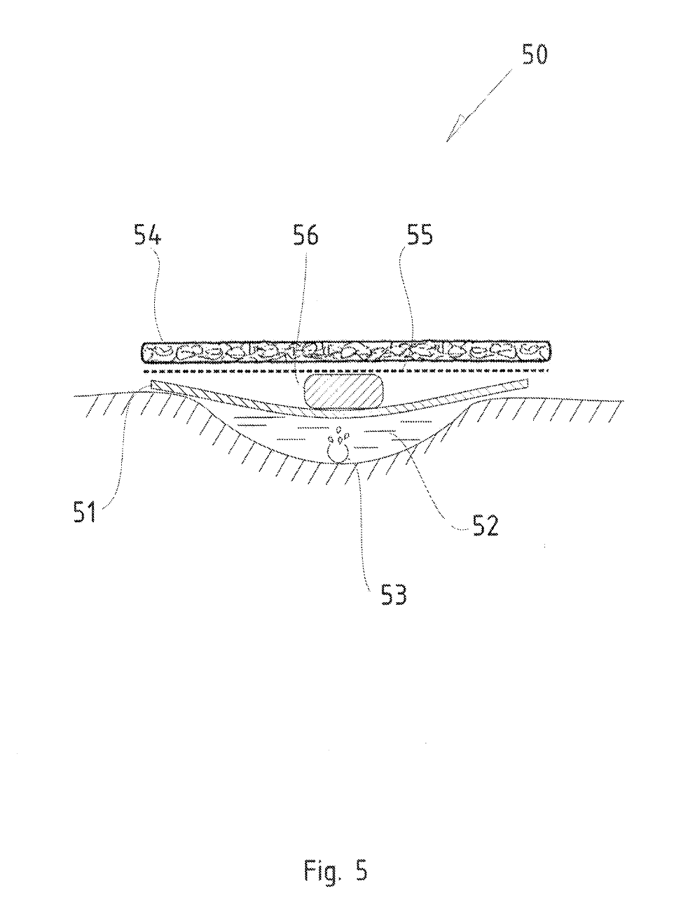

FIG. 5 shows a similar embodiment to FIG. 4. However, here in addition another pressurized cushion 56 is provided which is arranged between the primary wound dressing 51 and the diffusion barrier 55; however, it can also be arranged between the pressurized cushion 56 and the secondary wound dressing 54. The diffusion barrier can for example consist of modified cellulose (preferably CMC).

The pressurized cushion can for example be a fluid-proof packed cushion made of cellulose, likewise also for example a handle or a spherical object. In contrast to the configuration shown in FIG. 4 in this configuration along with or in place of the chemically and/or pharmacologically and/or physiologically acting active ingredient or active ingredient complex a physically acting active element can be realized as a hemostatic agent.

1. A wound care article exhibiting a quantity of super-absorbent polymers, characterized in that the wound care article exhibits at least one agent which can restrict the bleeding or the tendency to bleed.

2. The wound care article according to claim 1, characterized in that the agent is at least one chemically and/or pharmacologically and/or physiologically acting active ingredient or active ingredient complex.

3. The wound care article according to claim 1, characterized in that the agent is at least one physically acting active element.

4. The wound care article according to claim 1, characterized in that the wound care article
a) is present as an essentially flat material segment exhibiting absorption material, which is configured of an fleece with super-absorbent polymers distributed within as well as at least one chemically and/or pharmacologically and/or physiologically acting active ingredient or active ingredient complex;
b) is configured as or in combination with a tourniquet or compression dressing;
c) is configured as a combination of a primary, a non-absorbent or only marginally absorbent wound dressing which exhibits at least one chemically and/or pharmacologically and/or physiologically acting active ingredient or active ingredient complex, and a as well as a secondary wound dressing arranged peripheral from this primary wound dressing, which contains super-absorbing polymers, wherein if applicable a diffusion barrier is arranged between both wound dressings,
d) is configured in the form of a wound dressing packet, exhibiting a primary wound dressing with at least one chemically and/or pharmacologically and/or physiologically acting active ingredient or active ingredient complex as well as a compress segment arranged on the wound dressing which exhibits at least in segments super-absorbent polymers and/or
e) is configured as a material segment with a longitudinal extension exhibiting absorption material, wherein the material segment exhibits elastically deformable properties, and wherein the material segment exhibits super-absorbent polymers as well as if applicable at least one chemically and/or pharmacologically and/or physiologically acting active ingredient or active ingredient complex.

5. The wound care article according to claim 1, characterized in that the wound care article in addition exhibits at least one nutritive, at least one disinfecting or decontaminating and/or at least one protease inhibiting acting active ingredient or active ingredient complex.

6. The use of a wound care article according to claim 1 for chronic wounds which if applicable exhibit slight bleeding, wherein the wound dressing is brought into direct contact with the wound.

7. The use of a wound care article according to claim 1 for acute bleeding wounds, wherein the wound care article is configured as a tourniquet or compression dressing and/or can be integrated into one.

8. The use of a wound care article according to claim 1 for acute bleeding wounds, wherein the wound care article is arranged downstream of an arterial Spanish tourniquet.

9. The use of a wound care article according to claim 1 for traumatically produced wounds.

10. The use of a wound care article according to claim 1 for military purposes.

11. A kit for acute, emergency or military medical or chronic care, exhibiting a wound care article according to claim 1.

12. The use of a wound care article according to claim 1 for the treatment of wounds which, due to a disorder such as hemophilia and/or a systemically conditioned, pathologically and/or pharmacologically evoked coagulation deficit exhibit a deteriorated healing process.

13. The use of a wound care article according to claim 1 for operative or post-operative care.

14. The wound care article according to claim 2, characterized in that the agent is at least one physically acting active element.

15. The wound care article according to claim 2, characterized in that the wound care article
a) is present as an essentially flat material segment exhibiting absorption material, which is configured of an fleece with super-absorbent polymers distributed within as well as at least one chemically and/or pharmacologically and/or physiologically acting active ingredient or active ingredient complex;
b) is configured as or in combination with a tourniquet or compression dressing;
c) is configured as a combination of a primary, a non-absorbent or only marginally absorbent wound dressing which exhibits at least one chemically and/or pharmacologically and/or physiologically acting active ingredient or active ingredient complex, and a as well as a secondary wound dressing arranged peripheral from this primary wound dressing, which contains super-absorbing polymers, wherein if applicable a diffusion barrier is arranged between both wound dressings,
d) is configured in the form of a wound dressing packet, exhibiting a primary wound dressing with at least one chemically and/or pharmacologically and/or physiologically acting active ingredient or active ingredient complex as well as a compress segment arranged on the wound dressing which exhibits at least in segments super-absorbent polymers and/or
e) is configured as a material segment with a longitudinal extension exhibiting absorption material, wherein the material segment exhibits elastically deformable properties, and wherein the material segment exhibits super-absorbent polymers as well as if applicable at least one chemically and/or pharmacologically and/or physiologically acting active ingredient or active ingredient complex.

16. The wound care article according to claim 3, characterized in that the wound care article
a) is present as an essentially flat material segment exhibiting absorption material, which is configured of an fleece with super-absorbent polymers distributed within as well as at least one chemically and/or pharmacologically and/or physiologically acting active ingredient or active ingredient complex;
b) is configured as or in combination with a tourniquet or compression dressing;
c) is configured as a combination of a primary, a non-absorbent or only marginally absorbent wound dressing which exhibits at least one chemically and/or pharmacologically and/or physiologically acting active ingredient or active ingredient complex, and a as well as a secondary wound dressing arranged peripheral from this primary wound dressing, which contains super-absorbing polymers, wherein if applicable a diffusion barrier is arranged between both wound dressings,
d) is configured in the form of a wound dressing packet, exhibiting a primary wound dressing with at least one chemically and/or pharmacologically and/or physiologically acting active ingredient or active ingredient complex as well as a compress segment arranged on the wound dressing which exhibits at least in segments super-absorbent polymers and/or
e) is configured as a material segment with a longitudinal extension exhibiting absorption material, wherein the material segment exhibits elastically deformable properties, and wherein the material segment exhibits super-absorbent polymers as well as if applicable at least one chemically and/or pharmacologically and/or physiologically acting active ingredient or active ingredient complex.

17. The wound care article according to claim 2, characterized in that the wound care article in addition exhibits at least one nutritive, at least one disinfecting or decontaminating and/or at least one protease inhibiting acting active ingredient or active ingredient complex.

18. The wound care article according to claim 3, characterized in that the wound care article in addition exhibits at least one nutritive, at least one disinfecting or decontaminating and/or at least one protease inhibiting acting active ingredient or active ingredient complex.

19. The wound care article according to claim 4, characterized in that the wound care article in addition exhibits at least one nutritive, at least one disinfecting or decontaminating and/or at least one protease inhibiting acting active ingredient or active ingredient complex.

20. The wound care article according to claim 14, characterized in that the wound care article in addition exhibits at least one nutritive, at least one disinfecting or decontaminating and/or at least one protease inhibiting acting active ingredient or active ingredient complex.
